(19) United States
(12) Patent Application Publication
(10) Pub. No.: US 2007/0156106 A1
(43) Pub. Date: Jul. 5, 2007

(54) Disposable absorbent articles having temperature sensors

(76) Inventors: Thomas James Klofta, Cincinnati, OH (US); Robin Lynn McKiernan, Mason, OH (US); Edward Lawrence Schmidt, Liberty, OH (US); Darrell Ian Brown, Mason, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/589,414

(22) Filed: Oct. 30, 2006

Related U.S. Application Data
(60) Provisional application No. 60/756,237, filed on Jan. 3, 2006.

(51) Int. Cl. A61F 13/15

(52) U.S. Cl. 604/361

(57) ABSTRACT

A disposable absorbent article suitable for receiving and containing bodily exudates of a wearer where the article includes a front waist region, a back waist region and a crotch region disposed between the front and back waist regions, each region having both an interior surface and an exterior surface and wherein the article further includes a sensor comprising a material selected from the group consisting of thermochromic inks, thermochromic dyes, thermochromic liquid crystalline materials, and combinations thereof such that the sensor is fastened to an interior surface of any one of the regions.

### CROSS REFERENCE TO RELATED APPLICATION

This application claims the benefit of U.S. Provisional Application No. 60/756,237 filed Jan. 3, 2006.

### FIELD OF THE INVENTION

The present invention relates to a disposable absorbent article suitable for receiving and containing bodily exudates of a wearer, the article comprising a front waist region, a back waist region and a crotch region disposed between the front and back waist regions, each region having both an interior surface and an exterior surface and wherein the article further comprises a sensor comprising one or more materials selected from the group consisting of thermochromic liquid crystalline materials, thermochromic dyes, thermochromic inks, and combinations thereof such that the sensor is fastened under or above one or more interior surfaces of any one of the regions and contacts the wearer's skin when worn. In certain embodiments, the sensor may be attached to a flap that is attached to one or more surfaces of region of the absorbent article. For instance, this flap may include a polymeric film flap onto which the thermochromic liquid crystalline material is disposed. This flap may be moved or flipped from the inside to the outside of the article or vice versa.

### BACKGROUND OF THE INVENTION

One of the primary concerns of a parent or caregiver of an infant is the health and wellness of the infant. As such, there are a number of diagnostic tools that have been found useful to indicate the health and wellness of infants. These tools include thermometers, urinalysis test strips to measure pH and other properties, wetness indicators, etc. Oftentimes, however, these indicators are utilized primarily by professionals in clinical and health care environments rather than by caregivers. In order for these diagnostic devices to be convenient and cost effective for caregivers to use, Applicants therefore thought that it would be desirable to incorporate such indicators into the daily infant care routine. One effective way of including these indicators into the daily care routine of infants would be integration of sensors that include these indicators directly into the design of the disposable absorbent articles. In particular, the present invention is directed to providing a disposable absorbent article to a consumer for infant or adult use wherein the article includes a thermochromic liquid crystalline material, thermochromic dye, and/or thermochromic ink sensor that indicates a change in the body temperature of the infant, in particular to indicate to the caregiver the onset or present condition of an elevated temperature, especially in relation to the existence of the fever condition. This temperature sensor might also be employed to indicate unhealthy hypothermic temperatures or simply to monitor the ambient conditions of the user in order to insure their comfort or to simultaneously measure all these temperature conditions. This sensor may be disposed on one or more surfaces of the front waist, crotch, or back waist regions to allow for easy view of the sensor and the condition it is indicating. It might also be desirable to include a flap onto which the sensor is fastened allowing insertion of the flap on the inside of the article or employment of the flap for reading of the temperature on the outside of the article.

### SUMMARY OF THE INVENTION

The present invention relates to a disposable absorbent article suitable for receiving and containing bodily exudates of a wearer, the article comprising a front waist region, a back waist region and a crotch region disposed between the front and back waist regions, each region having both an interior surface and an exterior surface and wherein the article further comprises a sensor comprising one or more materials selected from the group consisting of thermochromic liquid crystalline materials, thermochromic dyes, thermochromic inks, and combinations thereof such that the sensor is fastened to one or more interior surfaces of any one of the regions and is in contact with the skin of the wearer when worn.

In another embodiment, the invention relates to a method of visually detecting a wearer's body temperature as well as ambient temperature conditions where the method comprises the steps of:
a. providing a caregiver with a disposable absorbent article suitable for receiving and containing bodily exudates of a wearer, the article comprising a front waist region, a back waist region and a crotch region disposed between the front and back waist regions, each region having an interior surface and an exterior surface and wherein the article further comprises a sensor comprising one or more materials selected from the group consisting of thermochromic inks, thermochromic dyes, thermochromic liquid crystalline materials, or combinations thereof such that the sensor is fastened to one or more interior surfaces of any one of the regions and is in contact with the skin of the wearer when worn;
b. donning the article onto a wearer; and
c. instructing the caregiver to visually monitor the wearer's health status via the appearance of the sensor.

Additionally, the present invention is directed to a kit for visually detecting a wearer's body temperature as well as ambient temperature conditions where the kit comprises:
a. one or more disposable absorbent articles suitable for receiving and containing bodily exudates of a wearer, each of the articles comprising a front waist region, a back waist region and a crotch region disposed between the front and back waist regions, each region having an interior surface and an exterior surface and wherein each of the articles further comprise a sensor comprising one or more materials selected from the group consisting of thermochromic inks, thermochromic dyes, thermochromic liquid crystalline materials, or combinations thereof such that the sensor is fastened to one or more surfaces of any one of the regions and is in contact with the skin of the wearer when worn;
b. a set of instructions for a caregiver or the wearer to visually detect the status of an infant wearer's health; and
c. a system for recording and tracking temperature of the infant wearer.

### BRIEF DESCRIPTION OF THE DRAWINGS

FIG. 1 is a plan view of an article made according to the present invention.

FIG. 2 is a front perspective view of the diaper of FIG. 1.

FIG. 3 is a back perspective view of the diaper of FIG. 1.

FIG. 4 is a perspective view of a disposable pant-type garment made according to the present invention.

FIG. 5 is a top perspective view of a sensor of the present invention.

FIG. 6 is a cross-sectional view of the sensor of FIG. 5.

FIG. 7 is a top perspective view of a bar shaped sensor which contains a plurality of thermochromic liquid crystalline material areas.

FIG. 8 shows a cross-sectional view of the sensor of FIG. 7.

FIG. 9 is a top perspective view of a sensor with a graphical indicia that changes upon exposure to a predetermined temperature of the wearer.

FIG. 10 is a top perspective view of a sensor with a linguistic indicator.

FIG. 11A is a front perspective view of a bar type sensor of the present invention where the graphical indicia indicate a normal skin temperature for a wearer of the article of the present invention.

FIG. 11B is a front perspective view of a bar type sensor of the present invention where the graphical indicia indicate an slightly elevated skin temperature for a wearer of the article of the present invention.

FIG. 11C is a front perspective view of a bar type sensor of the present invention where the graphical indicia indicate a feverish skin temperature for a wearer of the article of the present invention.

FIG. 12A is a front perspective view of a moving line bar type sensor of the present invention where the graphical indicia indicate a normal skin temperature for a wearer of the article of the present invention.

FIG. 12B is a front perspective view of a moving line bar type sensor of the present invention where the graphical indicia indicate a slightly elevated skin temperature for a wearer of the article of the present invention.

FIG. 12C is a front perspective view of a moving line bar type sensor of the present invention where the graphical indicia indicate a feverish skin temperature for a wearer of the article of the present invention.

FIG. 13 is a front perspective view of the sensor under the top sheet.

FIG. 14 is a front perspective view of an embodiment of a temperature sensor utilizing a color indicator;.

FIG. 15 is a front perspective view of an embodiment of a temperature sensor utilizing a plurality of regions and numeric indicators.

FIG. 16 is a front perspective view of a plurality of measurement regions that are physically separated from each other that can form a specific indicia.

FIG. 17 is a front perspective view of a plurality of measurement regions that are physically separated from each other that can form a specific indicia.

FIG. 18 is a front perspective view of a plurality of measurement regions that are physically separated from each other, wherein each region contains a formula with a different clearing temperature.

FIG. 19 is a front perspective view of a plurality of patterned bonds.

### DETAILED DESCRIPTION OF THE INVENTION

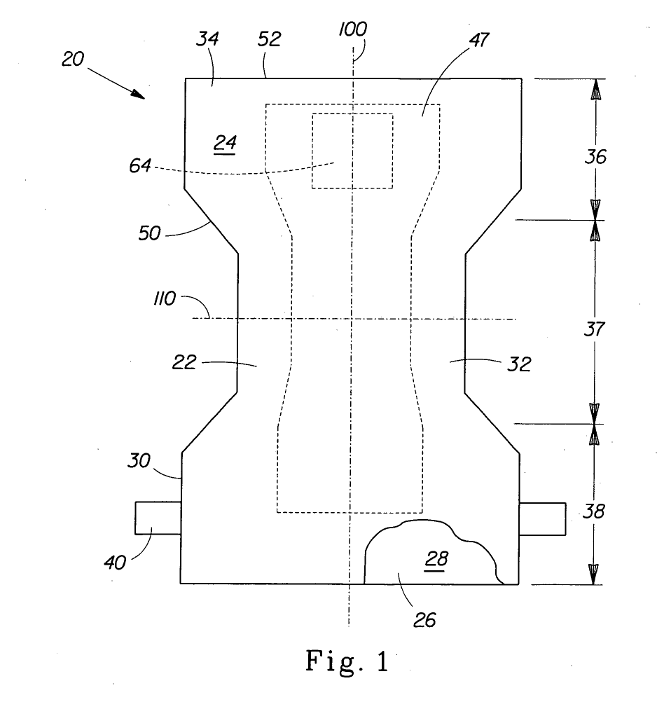

As used herein, the term “absorbent article” refers to devices which absorb and contain body exudates and, more specifically, refers to devices which are placed against or in proximity to the body of the wearer to absorb and contain the various exudates discharged from the body. The term “disposable” is used herein to describe absorbent articles which generally are not intended to be laundered or otherwise restored or reused as absorbent articles (i.e., they are intended to be discarded after a single use and, preferably, to be recycled, composted or otherwise discarded in an environmentally compatible manner). A “unitary” absorbent article refers to absorbent articles which are formed of separate parts united together to form a coordinated entity so that they do not require separate manipulative parts like a separate holder and/or liner. A preferred embodiment of an absorbent article of the present invention is the unitary disposable absorbent article, diaper 20, shown in FIG. 1. As used herein, the term “diaper” refers to an absorbent article generally worn by infants and incontinent persons about the lower torso. The present invention is also applicable to other absorbent articles such as training pants, swim pants, incontinence briefs, incontinence undergarments, absorbent inserts, diaper holders and liners, feminine hygiene garments, wipes, bandages and the like.

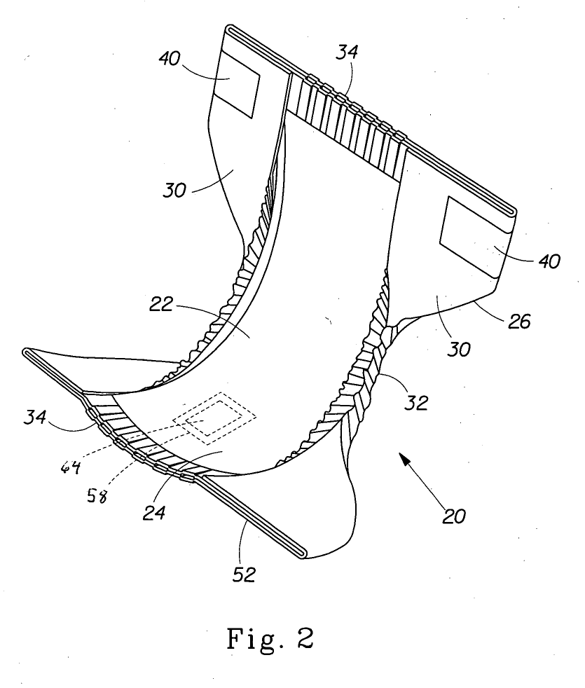

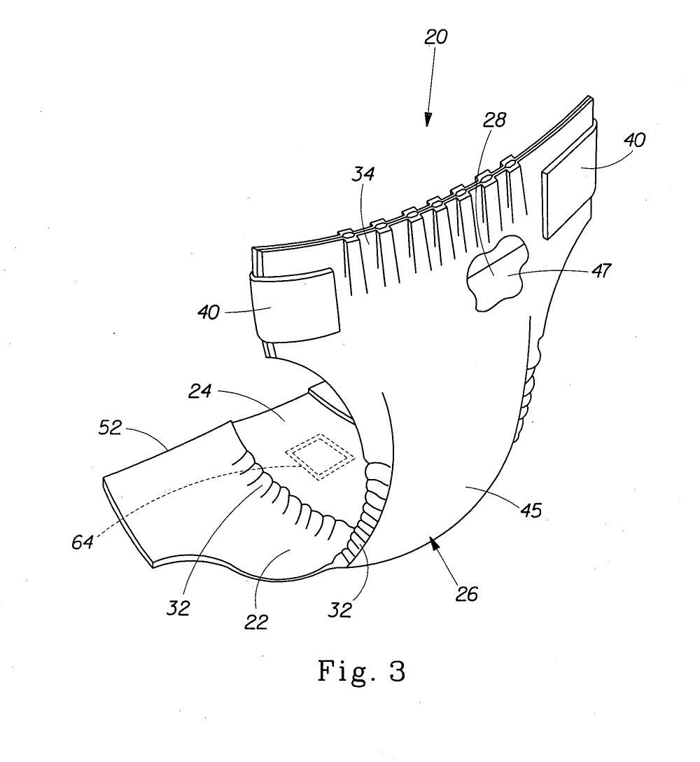

FIG. 1 is a plan view of the diaper 20 of the present invention in a flat-out state with portions of the structure being cut-away to more clearly show the construction of the diaper 20 that includes a sensor 64. The portion of the diaper 20 which faces the wearer is oriented towards the viewer. As shown in FIG. 1, the diaper 20 preferably comprises a topsheet 24; a backsheet 26; an absorbent core 28 which is preferably positioned between at least a portion of the topsheet 24 and the backsheet 26; side panels 30; elasticized leg cuffs 32; an elastic waist feature 34 ; and a fastening system generally designated 40. The diaper 20 is shown in FIG. 1 to have a front 20 waist region 36, a back waist region 38 opposed to the front waist region 36 and a crotch region 37 located between the front waist region 36 and the back waist region 38. Each of these three regions has an exterior surface (which faces the garment) and an interior surface (which faces the body of the wearer). The periphery of the diaper 20 is defined by the outer edges of the diaper 20 in which longitudinal edges 50 run generally parallel to the longitudinal centerline 100 of the diaper 20 and end edges 52 run between the longitudinal edges 50 generally parallel to the lateral centerline 110 of the diaper 20. FIGS. 2 and 3 merely show front and back perspective views of the diaper of FIG. 1.

The chassis 22 of the diaper 20 comprises the main body of the diaper 20. The chassis 22 comprises at least a portion of the absorbent core 28 and preferably an outer covering including the topsheet 24 and/or the backsheet 26. If the absorbent article comprises a separate holder and a liner, the chassis 22 generally comprises the holder and the liner. (For example, the holder may comprise one or more layers of material to form the outer cover of the article and the liner may comprise an absorbent assembly including a topsheet, a backsheet, and an absorbent core. In such cases, the holder and/or the liner may include a fastening element which is used to hold the liner in place throughout the time of use.) For unitary absorbent articles, the chassis 22 comprises the main structure of the diaper with other features added to form the composite diaper structure. While the topsheet 24, the backsheet 26, and the absorbent core 28 may be assembled in a variety of well known configurations, preferred diaper configurations are described generally in U.S. Pat. Nos. 3,860,003, 5,151,092, 5,221,274, 5,554,145, 5,569,234, 5,580,411, and 6,004,306.

The backsheet 26 is generally that portion of the diaper 20 positioned adjacent to the garment facing surface 45 of the absorbent core 28 which prevents the exudates absorbed and contained therein from soiling articles which may contact the diaper 20, such as bedsheets and undergarments. In preferred embodiments, the backsheet 26 is impervious to liquids (e.g., urine) and comprises a thin plastic film such as a thermoplastic film having a thickness of about 0.012 mm (0.5 mil) to about 0.051 mm (2.0 mils). Suitable backsheet films include those manufactured by Tredegar Corporation, based in Richmond, Va., and sold under the trade name CPC2 film. Other suitable backsheet materials may include breathable materials which permit vapors to escape from the diaper 20 while still preventing exudates from passing through the backsheet 26. Exemplary breathable materials may include materials such as woven webs, nonwoven webs, composite materials such as film-coated nonwoven webs, microporous films such as manufactured by Mitsui Toatsu Co., of Japan under the designation ESPOIR NO and by Tredegar Corporation of Richmond, Va. and sold under the designation EXAIRE., and monolithic films such as manufactured by Clopay Corporation, Cincinnati, Ohio, under the name HYTREL blend P18-3097. Some breathable composite materials are described in greater detail in PCT Application No. WO 95/16746 published on Jun. 22, 1995 in the name of E. I. DuPont; U.S. Pat. Nos. 5,938,648, 5,865,823, and 5,571,096.

The backsheet 26, or any portion thereof, may be elastically extensible in one or more directions. In one embodiment, the backsheet 26 may comprise a structural elastic-like film (“SELF”) web. A structural elastic-like film web is an extensible material that exhibits an elastic-like behavior in the direction of elongation without the use of added elastic materials and is described in more detail in U.S. Pat. No. 5,518,801. In alternate embodiments, the backsheet 26 may comprise elastomeric films, foams, strands, or combinations of these or other suitable materials with nonwovens or synthetic films.

The backsheet 26 may be joined to the topsheet 24, the absorbent core 28 or any other element of the diaper 20 by any attachment means known in the art. As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element. For example, the attachment means may include a uniform continuous layer of adhesive, a patterned layer of adhesive, or an array of separate lines, spirals, or spots of adhesive. One preferred attachment means comprises an open pattern network of filaments of adhesive as disclosed in U.S. Pat. No. 4,573,986. Other suitable attachment means include several lines of adhesive filaments which are swirled into a spiral pattern, as is illustrated by the apparatus and methods shown in U.S. Pat. Nos. 3,911,173, 4,785,996, and 4,842,666. Adhesives which have been found to be satisfactory are manufactured by H. B. Fuller Company of St. Paul, Minn. and marketed as HL-1620 and HL-1358-XZP. Alternatively, the attachment means may comprise heat bonds, pressure bonds, ultrasonic bonds, dynamic mechanical bonds, cohesive bonds, or any other suitable attachment means or combinations of these attachment means as are known in the art.

The topsheet 24 is preferably positioned adjacent to the body surface 47 of the absorbent core 28 and may be joined thereto and/or to the backsheet 26 by any attachment means known in the art. Suitable attachment means are described above with respect to means for joining the backsheet 26 to other elements of the diaper 20. In one preferred embodiment of the present invention, the topsheet 24 and the backsheet 26 are joined directly to each other in some locations and are indirectly joined together in other locations by directly joining them to one or more other elements of the diaper 20.

The topsheet 24 is preferably compliant, soft-feeling, and non-irritating to the wearer's skin. Further, at least a portion of the topsheet 24 is liquid pervious, permitting liquids to readily penetrate through its thickness. A suitable topsheet may be manufactured from a wide range of materials, such as porous foams, reticulated foams, apertured plastic films, or woven or nonwoven materials of natural fibers (e.g., wood or cotton fibers), synthetic fibers (e.g., polyester or polypropylene fibers), or a combination of natural and synthetic fibers. If the topsheet 24 includes fibers, the fibers may be spunbond, carded, wet-laid, meltblown, hydroentangled, or otherwise processed as is known in the art. One suitable topsheet 24 comprising a web of staple-length polypropylene fibers is manufactured by Veratec, Inc., a Division of International Paper Company, of Walpole, Mass. under the designation P-8.

Suitable formed film topsheets are described in U.S. Pat. Nos. 3,929,135, 4,324,246, 4,342,314, 4,463,045, and 5,006,394. Other suitable topsheets 24 may be made in accordance with U.S. Pat. Nos. 4,609,518 and 4,629,643. Such formed films are available from The Procter & Gamble Company of Cincinnati, Ohio as “DRI-WEAVE” and from Tredegar Corporation, based in Richmond, Va., as “CLIFF-T.”

Preferably, at least a portion of the topsheet 24 is made of a hydrophobic material or is treated to be hydrophobic in order to isolate the wearer's skin from liquids contained in the absorbent core 28. If the topsheet 24 is made of a hydrophobic material, preferably at least a portion of the upper surface of the topsheet 24 is treated to be hydrophilic so that liquids will transfer through the topsheet more rapidly. The topsheet 24 can be rendered hydrophilic by treating it with a surfactant or by incorporating a surfactant into the topsheet or by other surface modification techniques such as plasma, oxidation, and surface grafting chemistries. Suitable methods for treating the topsheet 24 with a surfactant include spraying the topsheet 24 material with the surfactant and/or immersing the material into the surfactant. A more detailed discussion of such a treatment and hydrophilicity is contained in U.S. Pat. Nos. 4,988,344 and 4,988,345. A more detailed discussion of some suitable methods for incorporating a surfactant in the topsheet 24 can be found in U.S. Statutory Invention Registration No. H1670. Alternatively, the topsheet 24 may include an apertured web or film which is hydrophobic. This may be accomplished by eliminating the hydrophilizing treatment step from the production process and/or applying a hydrophobic treatment to the topsheet 24, such as a polytetraflouroethylene compound like TEFLON™ or a hydrophobic lotion composition, as described below. In such embodiments, it is preferred that the apertures be large enough to allow the penetration of aqueous fluids like urine without significant resistance.

Any portion of the top sheet 24 may be coated with a lotion as is known in the art.

Examples of suitable lotions include those described in U.S. Pat. Nos. 5,607,760, 5,609,587, 5,635,191, 5,643,588, 5,968,025, and 6,716,441. The lotion may function alone or in combination with another agent as the hydrophobizing treatment described above. The topsheet 24 may also include or be treated with antibacterial agents, some examples of which are disclosed in PCT Publication No. WO 95/24173 entitled “Absorbent Articles Containing Antibacterial Agents in the Topsheet For Odor Control” which was published on Sep. 14, 1995 in the name of Theresa Johnson. Further, the topsheet 24, the backsheet 26 or any portion of the topsheet or backsheet may be embossed and/or matte finished to provide a more cloth like appearance.

The topsheet 24 may comprise one or more apertures to ease penetration of exudates therethrough, such as urine and/or feces (solid, semi-solid, or liquid). The size of at least the primary aperture is important in achieving the desired waste encapsulation performance. If the primary aperture is too small, the waste may not pass through the aperture, either due to poor alignment of the waste source and the aperture location or due to fecal masses having a diameter greater than the aperture. If the aperture is too large, the area of skin that may be contaminated by “rewet” from the article is increased. Typically, the aperture should have an area of between about 10 cm² and about 50 cm². The aperture preferably has an area of between about 15 cm² and 35 cm².

Further, the topsheet 24 may be fully or partially elasticated or may be foreshortened so as to provide a void space between the topsheet 24 and the core 28. Exemplary structures including elasticized or foreshortened topsheets are described in more detail in U.S. Pat. Nos. 4,892,536, 4,990,147, 5,037,416, and 5,269,775.

The absorbent core 28 may comprise any absorbent material which is generally compressible, conformable, non-irritating to the wearer's skin, and capable of absorbing and retaining liquids such as urine and other certain body exudates. The absorbent core 28 may be manufactured in a wide variety of sizes and shapes (e.g., rectangular, hourglass, “T”-shaped, asymmetric, etc.) and may comprise a wide variety of liquid-absorbent materials commonly used in disposable diapers and other absorbent articles such as comminuted wood pulp, which is generally referred to as airfelt. Examples of other suitable absorbent materials include creped cellulose wadding; meltblown polymers, including coform; chemically stiffened, modified or cross-linked cellulosic fibers; tissue, including tissue wraps and tissue laminates; absorbent foams; absorbent sponges; superabsorbent polymers; absorbent gelling materials; or any other known absorbent material or combinations of materials.

The configuration and construction of the absorbent core 28 may also be varied (e.g., the absorbent core(s) or other absorbent structure(s) may have varying caliper zones, hydrophilic gradient(s), a superabsorbent gradient(s), or lower average density and lower average basis weight acquisition zones; or may comprise one or more layers or structures). Exemplary absorbent structures for use as the absorbent core 28 are described in U.S. Pat. Nos. 4,610,678, 4,673,402, 4,834,735, 4,888,231, 5,137,537, 5,147,345, 5,342,338, 5,260,345, 5,387,207, and 5,625,222.

The diaper 20 may also include a sublayer disposed between the topsheet 24 and the backsheet 26. (As used herein, the term “disposed” is used to mean that an element(s) of the diaper is formed (joined and positioned) in a particular place or position as a unitary structure with other elements of the diaper or as a separate element joined to another element of the diaper.) The sublayer may be any material or structure capable of accepting, storing or immobilizing bodily exudates. Thus, the sublayer may include a single material or a number of materials operatively associated with each other. Further, the sublayer may be integral with another element of the diaper 20 or may be one or more separate elements joined directly or indirectly with one or more elements of the diaper 20. Further, the sublayer may include a structure that is separate from the core 28 or may include or be part of at least a portion of the core 28.

Suitable materials for use as the sublayer may include large cell open foams, macro-porous compression resistant nonwoven highlofts, large size particulate forms of open and closed cell foams (macro and/or microporous), highloft nonwovens, polyolefin, polystyrene, polyurethane foams or particles, structures comprising a multiplicity of vertically oriented looped strands of fibers, absorbent core structures described above having punched holes or depressions, and the like. (As used herein, the term “microporous” refers to materials which are capable of transporting fluids by capillary action. The term “macroporous” refers to materials having pores too large to effect capillary transport of fluid, generally having pores greater than about 0.5 mm in diameter and, more specifically, having pores greater than about 1.0 mm in diameter.) One embodiment of a sublayer includes a mechanical fastening loop landing element, having an uncompressed thickness of about 1.5 millimeters available as XPL-7124 from the 3M Corporation of Minneapolis, Minn. Another embodiment includes a 6 denier, crimped and resin-bonded nonwoven highloft having a basis weight of 110 grams per square meter and an uncompressed thickness of 7.9 millimeters which is available from the Glit Company of Wrens, Ga. Other suitable absorbent and nonabsorbent sublayers are described in U.S. Pat. Nos. 6,680,422 and 5,941,864. Further, the sublayer, or any portion thereof, may include or be coated with a lotion or other known substances to add, enhance or change the performance or other characteristics of the element.

The diaper 20 may also comprise at least one elastic waist feature 34 that helps to provide improved fit and containment. The elastic waist feature 34 is generally intended to elastically expand and contract to dynamically fit the wearer's waist. The elastic waist feature 34 preferably extends at least longitudinally outwardly from at least one waist edge of the absorbent core 28 and generally forms at least a portion of the end edge 52 of the diaper 20. Disposable diapers are often constructed so as to have two elastic waist features, one positioned in the first waist region 36 and one positioned in the second waist region 38. Further, while the elastic waist feature 34 or any of its constituent elements may comprise one or more separate elements affixed to the diaper 20, the elastic waist feature 34 may be constructed as an extension of other elements of the diaper 20, such as the backsheet 26, the topsheet 24, or both the backsheet 26 and the topsheet 24.

The elastic waist feature 34 may be constructed in a number of different configurations including those described in U.S. Pat. Nos. 4,515,595, 4,710,189, 5,151,092, and 5,221,274. Other suitable waist configurations may include waistcap features such as those described in U.S. Pat. Nos. 5,026,364 and 4,816,025.

The diaper 20 may also include a fastening system 40. The fastening system 40 preferably maintains the first waist region 36 and the second waist region 38 in a configuration so as to provide lateral tensions about the circumference of the diaper 20 to hold the diaper 20 on the wearer. The fastening system 40 preferably comprises a fastener such as tape tabs, cohesives, hook and loop fastening components, interlocking fasteners such as tabs & slots, buckles, buttons, snaps, and/or hermaphroditic fastening components, although any other known fastening means are generally acceptable. Some exemplary surface fastening systems are disclosed in U.S. Pat. Nos. 3,848,594, 4,662,875, 4,846,815, 4,894,060, 4,946,527, 5,151,092, and 5,221,274. An exemplary interlocking fastening system is disclosed in U.S. Pat. No. 6432098. The fastening system 40 may also provide a means for holding the article in a disposal configuration as disclosed in U.S. Pat. No. 4,963,140. The fastening system may also include primary and secondary fastening systems, as disclosed in U.S. Pat. No. 4,699,622 to reduce shifting of overlapped portions or to improve fit as disclosed in U.S. Pat. No. 5,242,436, 5,499,978, 5,507,736, and 5,591,152.

In certain embodiments, the article may be preformed by the manufacturer to create a pant-type diaper as shown in FIG. 4. The terms “pant” or “pant-type diaper”, as used herein, refers to disposable garments having a waist opening and leg openings designed for infant or adult wearers. A pant may be placed in position on the wearer by inserting the wearer's legs into the leg openings and sliding the pant into position about the wearer's lower torso. A pant may be preformed by any suitable technique including, but not limited to, joining together portions of the article using refastenable and/or non-refastenable bonds (e.g., seam, weld, adhesive, cohesive bond, fastener, etc.). A pant may be preformed anywhere along the circumference of the article (e.g., side fastened, front waist fastened). While the term “pant” is used herein, pants are also commonly referred to as “closed diapers”, “prefastened diapers”, “pull-on diapers”, “training pants”, “swim pants”, and “diaper-pants”. Suitable pants are disclosed in U.S. Pat. Nos. 5,246,433, 5,569,234, 6,120,487, 6,120,489, 4,940,464, 5,092,861, U.S. patent application Ser. No. 10/171,249, entitled “Highly Flexible And Low Deformation Fastening Device”, filed on Jun. 13, 2002; U.S. Pat. Nos. 5,897,545 and 5,957,908.

The diaper 20 may also comprise side panels 30. The side panels 30 may be elastic or extensible to provide a more comfortable and contouring fit by initially conformably fitting the diaper 20 to the wearer and sustaining this fit throughout the time of wear well past when the diaper 20 has been loaded with exudates since the elasticized side panels 30 allow the sides of the diaper 20 to expand and contract. The side panels 30 may also provide more effective application of the diaper 20 because even if the diaperer pulls one elasticized side panel 30 farther than the other during application, the diaper 20 will “self-adjust” during wear.

While the diaper 20 of the present invention preferably has the side panels 30 disposed in the second waist region 38, the diaper 20 may be provided with side panels 30 disposed in the first waist region 36 or in both the first waist region 36 and the second waist region 38. The side panels 30 may be constructed in any suitable configurations. Examples of diapers with elasticized side panels are disclosed in U.S. Pat. Nos. 4,857,067, 4,381,781 4,938,753, 5,151,092, 5,221,274, 5,669,897, and 6,004,306.

The diaper 20 preferably further includes leg cuffs 32 which provide improved containment of liquids and other body exudates. Leg cuffs 32 may also be referred to as leg bands, side flaps, barrier cuffs, or elastic cuffs. U.S. Pat. No. 3,860,003 describes a disposable diaper which provides a contractible leg opening having a side flap and one or more elastic members to provide an elasticized leg cuff (a gasketing cuff). U.S. Pat. Nos. 4,808,178 and 4,909,803 describe disposable diapers having “stand-up” elasticized flaps (barrier cuffs) which improve the containment of the leg regions. U.S. Pat. Nos. 4,695,278 and 4,795,454 describe disposable diapers having dual cuffs, including gasketing cuffs and barrier cuffs. In some embodiments, it may be desirable to treat all or a portion of the leg cuffs 32 with a lotion, as described above.

Embodiments of the present invention may also include pockets for receiving and containing waste, spacers which provide voids for waste, barriers for limiting the movement of waste in the article, compartments or voids which accept and contain waste materials deposited in the diaper 20, and the like, or any combinations thereof. Examples of pockets and spacers for use in absorbent products are described in U.S. Pat. Nos. 5,514,121, 5,171,236, 5,397,318, 5,540,671, 6,168,584, 5,306,266, and 5,997,520. Examples of compartments or voids are disclosed in U.S. Pat. Nos. 4,968,312, 4,990,147, 5,062,840, and 5,269,755. Examples of suitable transverse barriers are described in U.S. Pat. Nos. 5,554,142 and 5,653,703. Examples of other structures especially suitable for management of low viscosity feces are disclosed in U.S. Pat. Nos. 5,941,864, 5,977,430, and 6,013,063.

As shown in FIGS. 1-3 in diaper 20 of the present invention, a sensor 64 is provided to enable an indication of a condition of the wearer of the article. The sensor 64 is disposed and fastened above or below one or more interior surfaces of the front waist, back waist, or crotch regions. The sensors of the present invention may be placed inside the article such that the sensor is in contact with the wearer's abdomen, hip, or groin area, preferably the abdomen.

In certain embodiments, however, it is foreseeable that the sensor is visible on the exterior surface of the region in which the sensor is disposed. This visibility may be provided by a cut out, pocket, or window through that particular region of the article. Here, the window may provide the additional benefit of enhancing the readability of the sensor through magnification or other optical enhancement effects. In addition, the window may possess insulating properties to prevent the outside ambient temperatures from adversely influencing the temperatures measured on the skin.

Additionally, the sensor may come in a variety of shapes including, but not limited to, a shape selected from the group consisting of a circle, a partial circle, square, ellipse, rectangle, triangle, elongated strip, a non-geometric shape, and combinations thereof. In addition, the sensor may be uniquely colored and designed to coordinate and fit with the colors and design of the absorbent article. As noted previously, it may be convenient for the sensor to be disposed on or attached to an optional flap to give the caregiver the option of using the sensor within the article to measure core body temperatures, externally to measure ambient conditions, or completely removable to measure temperatures of any other animate or inanimate object. This flap may comprise the same materials employed for the backsheet and/or topsheet and may be shaped in a variety of configurations including, but not limited to, a semi-circle, a rectangle, a triangle, a semi-ellipse, etc. Finally, the sensor may be configured to simultaneously measure both the ambient external temperature as well as body temperature of the wearer.

The sensor may be fastened to the article above or below one or more of the interior surfaces of the front waist, back waist, or crotch regions. The topsheet may provide opportunity for placement on any of these interior surfaces. The sensor is fastened to one or more of these interior surfaces by a method selected from the group consisting of adhesion, hook and loop fastening, sewing, clamping, magnetism, binding by an outer layer, heat sealing, ultrasonic bonding, and combinations thereof. In certain cases, the sensor may be removably fastened, e.g., via the hook and loop fastening, reusable adhesives, cohesives, tab and slot fasteners, snap fasteners. etc. Where adhesives are used, they may be pressure activated adhesives or tapes and heat activated adhesives.

The sensor of the present invention comprises one or more materials selected from the group consisting of thermochromic liquid crystalline materials, thermochromic dyes, thermochromic inks, and combinations thereof. The materials are intended to serve as temperature indication mechanisms within the sensor. As used herein “thermochromic” means materials/inks/dyes that change their reflected color as a function of temperature. Suitable thermochromic liquid crystalline materials may be either temperature sensitive or temperature insensitive and chiral or cholesteric in nature. Suitable chiral or cholesteric thermochromic liquid crystalline materials may be like those incorporated into patches sold by Hallcrest Incorporated of Glenview, Ill., Kaz Inc. of Hudson, N.Y., Liquid Crystal Resources, LLC of Glenview, Ill., Medical Indicators of Pennington, N.J., and/or Thermographic Measurements of Flintshire, UK. Thermochromic dyes that can change color are typically called leuco dyes and such can be directly mixed in films, nonwovens, and elastics. Lueco dyes are commercially available from HWSands Corp. of Jupiter, Fla., and Color Change Corp. of Streamwood, Ill. Suitable thermochromic inks are commercially available from Chromatic Technologies, Inc. under the tradename Dynacolor® as body temperature or high temperature inks or from Sun Chemical's AIC subsidiary of France under the name ThermaSOFT®. Additional suitable thermochromic inks are detailed in U.S. Pat. Nos. 4,121,011, 4,826,550, 5,389,093, and 5,221,228. The thermochromic materials used can be in the form of fine pigments particles, microencapsulated materials, molecular materials and the like.

The one or more materials may be is applied in an application method selected from the group consisting of spraying, printing, coating, ultraviolet printing, painting, and combinations thereof. Suitable printing methods include, but are not limited to gravure, flexo, inkjet, slot, and screen printing.

In certain instances, the temperature insensitive thermochromic liquid crystalline material is colored at room temperature and normal human body temperatures and changes to clear in appearance in response to a noticeable increase in human body temperature. However, there are other suitable types of thermochromic liquid crystalline materials (temperature sensitive) that turn from colorless to red to orange to yellow to green to blue to violet and then back to colorless as the temperature is increased.

The thermochromic liquid crystalline material may be incorporated into a multi-layered sensor beneath which is disposed an underlayment layer 58. In most instances, this layer comprises a polymeric material selected from the group consisting of polyolefins (e.g. polyethylene or polypropylene), polyesters (e.g. polyethylene terephthalate or glycol modified polyethylene terephthalate), polyvinyls (e.g. polyvinylchloride or polyvinylidene chloride) or a combination thereof (e.g. polyvinylidene chloride coated polyethylene terephthalate). This underlayment layer is typically printed black to enhance the appearance of the thermochromic liquid crystalline material phase transformations but this underlayment layer may also may be printed to be colored such that this underlayment layer becomes visible in the sensor through the thermochromic liquid crystalline material as it becomes transparent or translucent in appearance. Alternatively, the underlayment layer may also comprise photochromic ink. Photochromic inks change color in response to the presence of ultraviolet or other wavelengths of radiation. In most cases, the photochromic inks change from invisible or clear to a humanly perceivable color upon exposure to a particular range of wavelengths.

As an additional component, the sensor may also comprise an overlayment layer 61 that is at least partially transparent or translucent such that the thermochromic liquid crystalline material is visible through the translucent or transparent overlayment portion. Suitable materials for this layer include those described herein as suitable for the underlayment layer. This overlayment layer may serve to contain the thermochromic liquid crystalline material, serve as a thermoconductive or insulating material for the sensor on the skin of a wearer, or serve as a contributor to enhance the appearance of the indicator that is placed on the face of the sensor.

For instance, the overlayment or underlayment layer may be printed to form one or more indicia 63 in the article. These indicia are selected from the group consisting of linguistic indicia (FIG. 10), graphical indicia (FIG. 9), numerical indicia (FIG. 15), color indicia (FIG. 14), and combinations thereof. The numerical indicia might provide an exact numerical read-out of the skin or ambient temperatures. The linguistic indicia provide some written signal to the caregiver that a change in condition has been detected. For instance, a suitable linguistic indicator includes the phrases “check me”, “fever”, “warm me up”, “I'm warm”, “I'm cold”, “check temp”, “check my temperature”, “OK”, etc. For instance, FIG. 10 depicts a sensor wherein the liquistic indicia changes from “OK” to “CHECK ME” when the requisite temperature change is indicated. A graphical indicia (e.g. FIG. 9) provides a pictorial depiction that serves as a signal to the caregiver that a change in condition has been detected in the body temperature of the wearer. For example, a graphical indicia envisioned by Applicants include the appearance of the following images in the event a high temperature is detected by the sensor: a sad or frowning face, a thermometer, a face with a thermometer, a single flame, a checkmark, a bold “X” mark, etc. A hypothermic condition might be graphically suggested with an icicle, blanket or other suitable graphical image. A color indicia (e.g. FIG. 14) is merely a change in color of the sensor that is easily noticeable by a caregiver wherein the change in color appears when an abnormally high or low temperature is detected by the sensor. For instance, the sensor may change from black to green in color, from clear to black, from red to clear, from green to black, from green to clear, etc.

In one embodiment the thermochromic liquid crystalline material may be encapsulated within gelatin microcapsules. This transport mechanism allows for a minimization in the amount of overall liquid crystalline material that is required within the sensor. The gelatin microcapsules also serve to protect the liquid crystalline material from environmental contamination.

Caregivers need sufficient time between the instance of removing the sensor from the skin of the infant or wearer and the instance of reading the body temperature of the wearer from the sensor before the indicator changes as a result of being removed from the wearer's body and influenced by the ambient conditions (i.e., the environment of the wearer and/or user). This can be accomplished through either hysteresis or delay. As used herein, “hysteresis” refers to the characteristic where the transition temperature from a transparent thermochromic liquid crystalline material to a color reflecting thermochromic liquid crystalline material as temperature decreases is lower than the transition temperature from a color reflecting thermochromic liquid crystalline material to a transparent thermochromic liquid crystalline material as temperature increases. The hysteresis characteristic of the material delays the temperature at which the transition from the transparent phase to color reflecting phase occurs.

The skin temperature as measured by the thermochromic liquid crystalline material sensor may also be locked for longer periods of time by applying a layer of material to the top, bottom or both regions of the structure. For instance, the sensor may be modified to exhibit a delay in temperature change of about 1 second, 5 seconds, 10, seconds, 30 seconds, 60 seconds, 90 seconds, or even about 120 seconds. Specifically, the top and bottom layers of material should be thermally capacitive, such as an elastomeric material like Exxon's Vista Maxx® with entrained low meltpoint waxes like Licowax PP230 and ChevTex 128 (commercially available from Clariant and Renkert Oil, respectively). These materials are polymers with an entrained low meltpoint wax, capable of absorbing or releasing significant amounts of heat energy before increasing or decreasing in temperature. Suitable polymer materials are disclosed in U.S. application Ser. Nos. 11/042236 and 11/042237, both filed on Jan. 25, 2005 in the name of J. P. Autran and both entitled “Fibers and Nonwovens Comprising Polypropylene Blends.”

For sensors made using temperature insensitive thermochromic liquid crystalline material, shearing of the sensor (e.g. bending) can cause a premature return to the original (i.e. low temperature) color. To prevent this, the sensor can be stiffened to prevent shearing. This can be accomplished through the use of a stiff underlayment layer such as 10 mil polyethylene terepthalate. This can also be accomplished through the use of stiffening agents such as adhesive applied either to the underside of the underlayment layer behind the liquid crystal or directly to the diaper area where the sensor will be subsequently bonded.

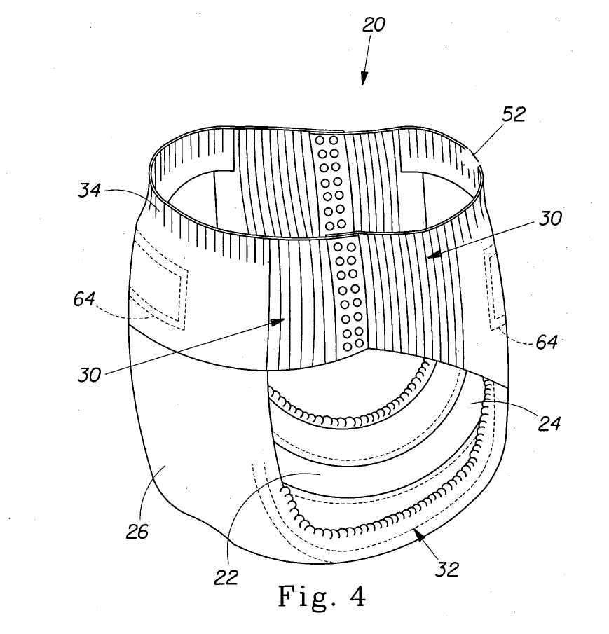

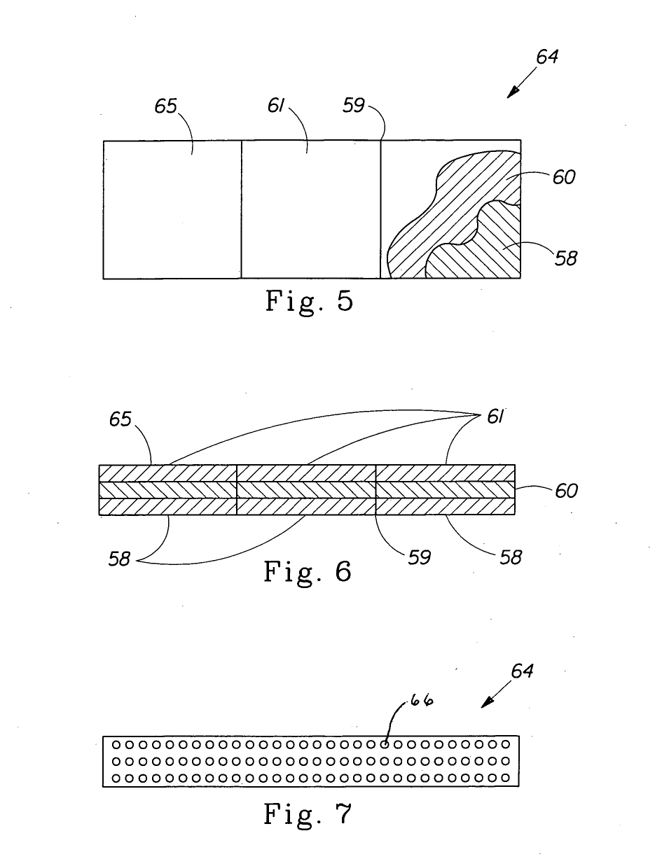

In some embodiments (e.g., FIGS. 5, 6, 11), the sensor comprises at least a first and a second material, whether thermochromic ink, thermochromic dye, or thermochromic liquid crystalline material, in a respective first and second measurement region wherein the first and second materials are physically separated from one another within the sensor by a separator. It is envisioned that there may be additional materials and corresponding measurement regions. Such an embodiment could be useful in the instance where, for example, a first measurement region comprising the first thermochromic liquid crystalline material was positioned toward the left end of the sensor. This first material turns a particular color, e.g., green, at normal human body temperatures (e.g., 96° F. to 100° F.). The second material would be positioned toward the middle of the sensor in a second measurement region. This second material changes color at temperatures greater than about 106° F. and the corresponding area of the sensor that it covers is then further overlaid with an overlayment that is printed with a graphical or linguistic indicator. Additional measurement regions within the sensor may be carved out as well that include additional thermochromic liquid crystalline materials, thermochromic dyes, thermochromic inks or combinations thereof. For instance, the above-described sensor may include a third thermochromic liquid crystalline material that changes color at temperatures greater than about 101° F. This third area in the sensor may comprise a similar overlayment layer that is printed with an indicator to signal the caregiver of a fever condition in the wearer. FIG. 5 depicts a top plan view of this three area sensor 64 that is useful in the article of the present invention. FIG. 6 shows a cross-sectional view of the sensor 64 of FIG. 5. The overlayment layer 61 is disposed on top of the thermochromic liquid crystalline material 60 and the underlayment layer 58 is disposed beneath the crystalline material 60. The areas of the sensor are distinguished from one another by one or more separators 59 that serve as barriers between the different types of thermochromic liquid crystalline materials within a sensor 64.

Another embodiment of a sensor (FIG. 15) is that with a plurality of regions. According to this embodiment, the sensor 64 includes a plurality of measurement regions 66 that are physically separated from each other. Each of the measurement regions include a material 67, such as a thermochromic ink, thermochromic dye, thermochromic liquid crystalline material, or a combination thereof. The materials may be selected so as to permit a range of temperatures to be detected, and to permit the temperatures to be discerned to within a range of precision. So that the caregiver may interpret the results, the sensor also includes information, in the form of alphanumeric characters, for example, displayed in certain areas 68 of the sensor. As illustrated, the sensor may be useful over a range of temperatures from 94-105° F., in increments of 0.2° F. The range of precision is also ±0.2° F., in so far as it is not possible to discern gradations in temperature of less than 0.2° F.

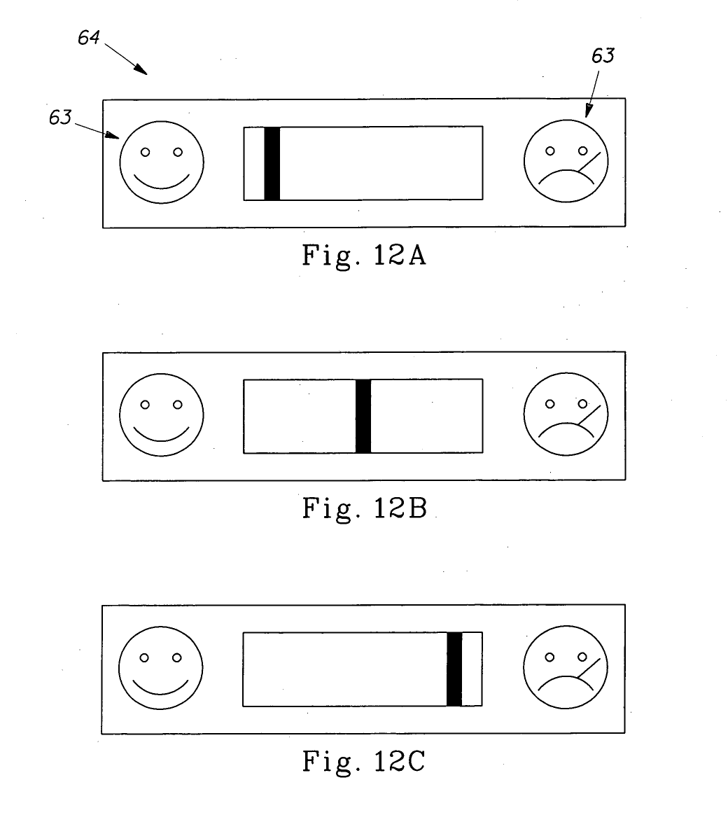

Another embodiment that is envisioned within the purview of this invention is the incorporation of a moving line thermochromic liquid crystalline material sensor (FIG. 12). This type of thermochromic liquid crystalline material temperature sensor is commercially sold to monitor the water temperature within aquariums by sticking this type of sensor to the outside surface of the aquarium. The moving line thermochromic liquid crystalline sensor is a band of multiple colors that moves across a scale as the detected temperature changes. For instance, the band of color may traverse the visible light spectrum with the leading edge usually violet and the trailing edge usually red in color or vice versa with the leading edge being red and the-trailing edge being violet. Alternatively, the band of color may exhibit other color combinations. The most effective background to enhance the visibility of the band of colors is black although other background colors can be used. The band of color can be designed to move vertically, horizontally, or at any angle in between. As the temperature increases, the band can move from lower to high on the appropriate axis.

Moreover, as mentioned earlier, the article may include a plurality of sensors within one or more article regions. For instance, the sensors may be numerous in the shape of a elongated strip formed from 2 mm diameter sensors spaced a short distance from one another in a 3 sensor dot by 30 sensor dot matrix, and this strip of sensors is present in the front waist region, particularly in the plane of the landing zone. FIG. 7 depicts this bar type sensor which contains a plurality of thermochromic liquid crystalline material areas. These crystalline material areas may all comprise the same material or may include a plurality of crystalline materials that are disposed within distinct regions of the sensor.

In one embodiment, the sensor may be fastened onto a flap which is disposed on an interior surface of either the front waist or rear waist region of the article such that the temperature of the wearer as well as the environment outside of the diaper may be monitored.

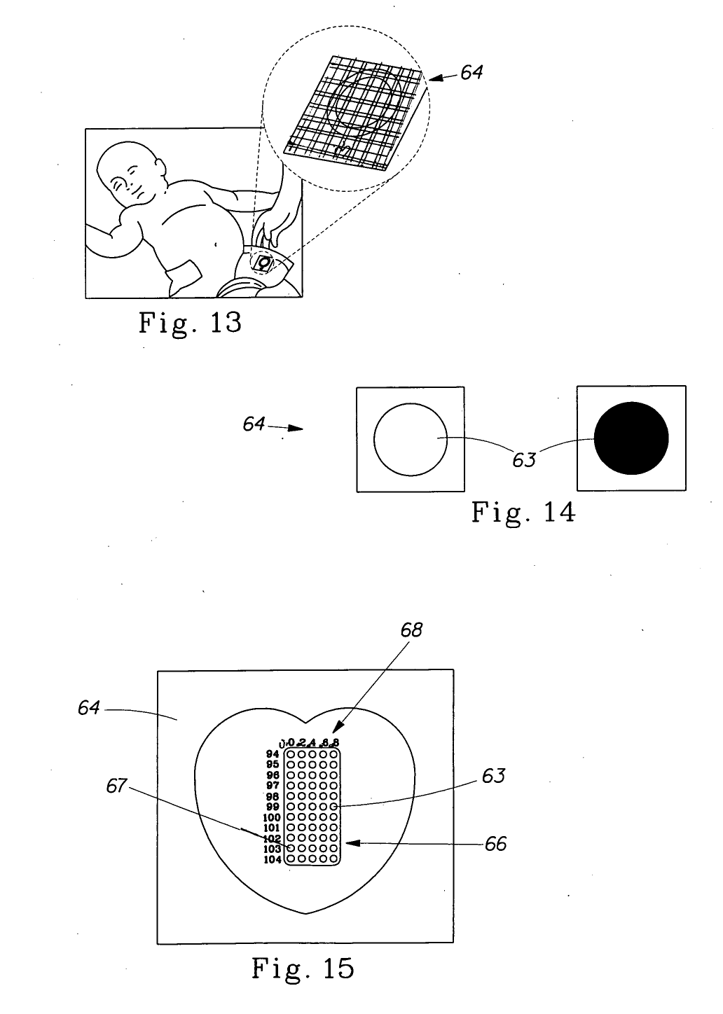

In one embodiment (FIG. 13), the sensor may be covered by a thin film or nonwoven. This further protects the skin of the wearer from irritation from the sensor and helps prevent the wearer from being able to remove the sensor. The film should ideally be thin and clear such as polyethylene. The nonwoven should be thin as well. For films and nonwovens where they are not clear or transparent, a hole or an array of holes (or apertures) can be used to allow the sensor to be viewed through the covering.

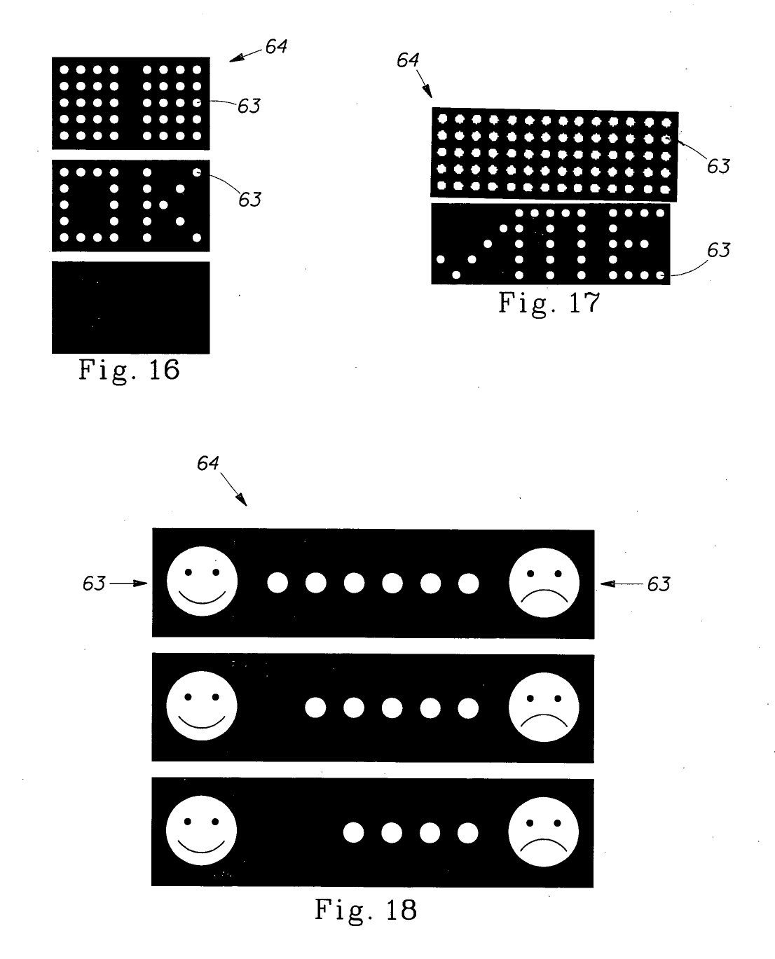

In another embodiment, the present invention may have a plurality of measurement regions that are physically separated from each other. Each of the measurement regions include a material, such as a thermochromic ink, thermochromic dye, thermochromic liquid crystalline material, or a combination thereof. The sensor may contain two different formulas with two different clearing temperatures. At room temperature all of the regions are one color (e.g. green). As the sensor is warmed to body temperature, the lower clearing regions clear and expose the background color (e.g. black). The regions that remain the first color (e.g. green) can form an indicia such as “OK” to reassure the caregiver that the sensor is working. As the sensor is further warmed to elevated body temperature, the higher clearing regions also clear and the entire sensor appears the background color (e.g. black) (FIG. 16). Alternatively, as the sensor is warmed to elevated body temperature, the lower clearing regions clear and expose the background color (e.g. black). The regions that remain the first color (e.g. green) can form an indicia such as “Check Me” to encourage the caregiver to check the baby's temperature (FIG. 17).

In another embodiment, the present invention may have a plurality of measurement regions as described above. Each measurement region may contain a formula with a different clearing temperature. Going from left to right, the clearing point is sequentially increased by, for example, in increments of 1 ° F. At room temperature all of the regions may be one color (e.g. green). As the sensor is warmed, the regions clear (sequentially from left to right) and expose the background color (e.g. black) (FIG. 18).

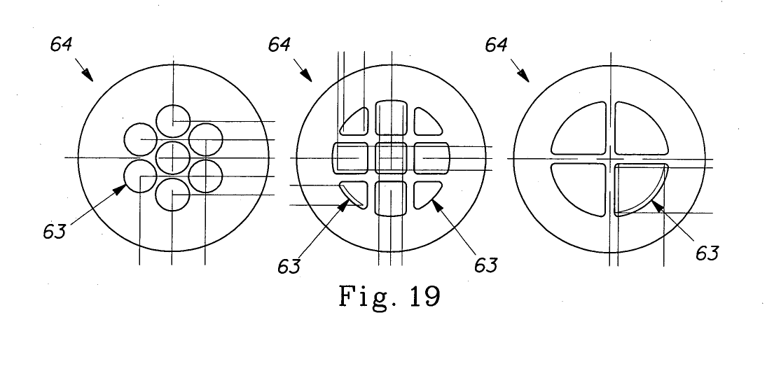

In another aspect, the present invention may use patterned bonds (FIG. 19) through a pattern heat seal bond to create wells to hold the material (e.g. liquid crystal) and prevent movement of the liquid.

In addition, the sensor of the presently claimed article may also be useful for detecting more than one condition. For instance, the sensor may contain multiple thermochromic liquid crystalline materials for detection of wearer temperature and environment conditions, the sensor may contain other indicators capable of measuring skin pH, microorganism content on the skin of a wearer, etc. Likewise, the article of the present invention may comprise additional sensors that perform these same additional functions.

The article of the present invention may be delivered to a consumer in a variety of forms. One manner for delivery is in the form of a kit for visually detecting an infant's health status. This kit comprises: a) one or more disposable absorbent articles suitable for receiving and containing bodily exudates, the article comprising a front waist region, a back waist region and a crotch region disposed between the front and back waist regions, each region having two opposing longitudinal edges, and wherein the article further comprises a window on any one of the regions for viewing a fastened sensor placed onto an interior surface of one or more of the regions and b) one or more sensors fastened to an interior surface of one or more of the regions wherein the sensors are selected from the group consisting of temperature sensors, wetness sensors, dehydration sensors, rash sensors, urinalysis test strips, pH sensors, and combinations thereof. In another embodiment, the kit may comprise a tracking sheet or internet web site input to record readings from the sensor. In addition, the tracking sheet or web site may provide additional information to enable the caregiver to properly care or treat the particular condition. Even these embodiments may comprise a sensor that may be removably fastened onto the interior surface of the article.

The present invention further relates to various methods of using the articles of the invention. For instance, one method is that of visually detecting an infant's health status or potential health issues, wherein the method comprises the steps of: a) providing a caregiver with a disposable absorbent article suitable for receiving and containing bodily exudates of a wearer, the article comprising a front waist region, a back waist region and a crotch region disposed between the front and back waist regions, each region possessing an interior surface and an exterior surface and wherein the article further comprises a sensor comprising one or more materials selected from the group consisting of thermochromic liquid crystalline materials, thermochromic inks, thermochromic dyes, and combinations thereof such that the sensor is fastened to an interior surface of any one of the regions; b) donning the article onto a wearer; and c) instructing the caregiver to visually monitor the wearer's health status via the appearance of the sensor.

In another aspect, the present invention may be in the form of a kit for visually detecting and monitoring a wearer's health status, the kit comprising: a) one or more disposable absorbent articles suitable for receiving and containing bodily exudates of a wearer, each of the articles comprising a front waist region, a back waist region and a crotch region disposed between the front and back waist regions, each region an interior surface and an exterior surface and wherein each of the articles further comprise a sensor comprising one or more materials selected from the group consisting of thermochromic liquid crystalline materials, thermochromic inks, thermochromic dyes, and combinations thereof such that the sensor is fastened to an interior surface of any one of the regions; b) a set of instructions for a caregiver or the wearer to visually detect the status of a wearer's health; and optionally, c) a system for recording and tracking temperature of the infant wearer. This system may comprise recording and tracking methods selected from the group consisting of hard-copy systems, computer input/output systems, web-based input/output, and combinations thereof. A caregiver's input of their child's temperature data would be an especially attractive means to teach the caregiver about the signs of high (fever) or low body (hypothermic) temperatures and potential treatments. In addition, if ambient environmental temperature monitoring was employed, the caregiver could be taught the importance of maintaining comfortable ambient temperatures to enhance sleep as well as to avoid such dangerous conditions such as SIDS or overheated internal car temperatures which are typical during the summer.

### EXAMPLES

### Example 1

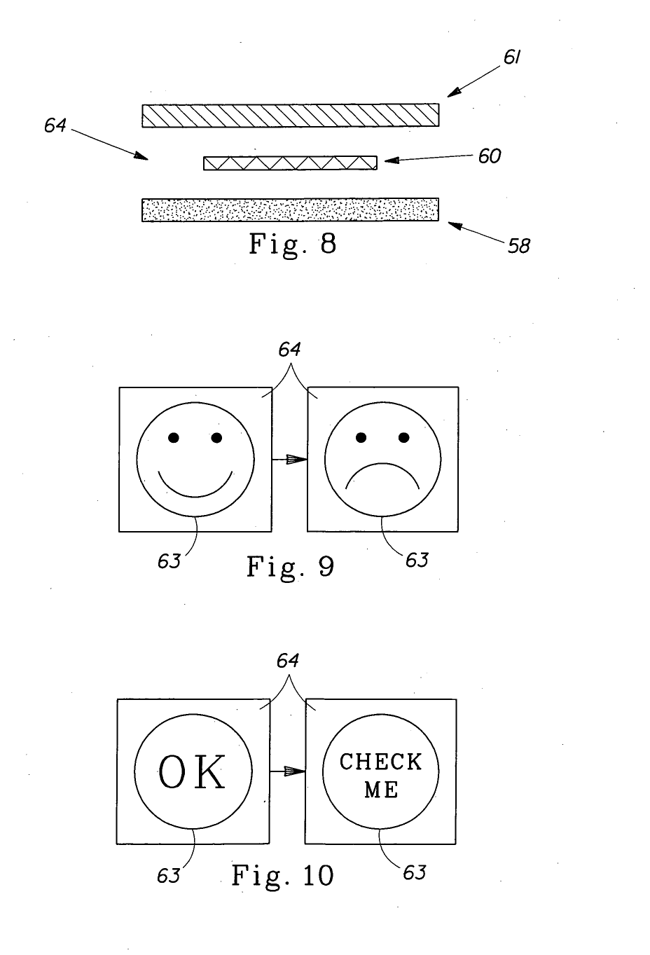

An absorbent article of the present invention is prepared by providing a diaper chassis as disclosed in any one of U.S. Pat. Nos. 3,860,003, 4,636,207, 4,695,278, 4,704,115, 4,795,454, 4,900,317, 4,909,803 (Reissued as USRE34920), U.S. Pat. Nos. 5,085,654, 5,492,751, 6,476,288, 6,627,787, 5,507,760, 5,609,587, 5,635,191, 5,643,588, 6,118,041 and SIR H1630. The sensor is a thermochromic liquid crystal article that is made up of three layers; a substrate, the thermochromic liquid crystalline material and an overlayment layer as shown in FIG. 8. In certain instances, the sensor may be rectangular in shape, having a width of approximately 2.5 cm. and a length of approximately 2.5 cm. However, other shapes are envisioned herein like rectangular with rounded corners, circular, oval, etc. The sensor may range in area from about 2 cm² to about 25 cm². For instance, the sensor of this example may be about 6.25 cm² in area. The underlayment layer and the overlayment layer may be made from polyester films. Preferably the underlayment is made of glycol modified polyethylene terephalate (e.g. as available from Klöckner Pentaplast, Montabaur, Germany) having a thickness of from about 1 to 10 mils (25 to 250 um). Preferably the overlayment is made of polyvinylidene chloride coated polyethylene terephalate (e.g. as available from Dupont Teijin Films) having a thickness of from about 0.5 to 2 mils (12.5 to 50 um). The underlayment and overlayment layers may be made with other polymeric materials, however, and in such instances may exhibit a thickness of up to about 250 um. The underlayment layer may comprise a black-colored polymeric sheet or may be clear and painted on the underside with a black paint applied thick enough to create a black opaque appearance. The underlayment may also be colored any other color such as navy blue, green, teal, purple, etc. The thermochromic liquid crystalline material is commercially available from Liquid Crystal Resources, LLC as “Unsealed Cholesteric Liquid Crystal Clearing Point Formulation with Hysteresis”. The resulting thermochromic liquid crystalline material is temperature insensitive formula and is coated onto the underlayment layer via a slot coater. The thermochromic liquid crystalline material exhibits the follow characteristics upon application to the underlayment layer:
1. Type—cholesteric compounds, temperature insensitive (aka “clearing point” liquid crystalline materials)
2. Preparation of thermochromic liquid crystalline material—Not encapsulated
3. Application thickness—greater than about 50 um (2 mil)
4. Trigger temperature—about 37.8° C. or 100.0° F.
5. Accuracy—about ±0.1° C. (0.2° F.)
6. Repeatability—less than about ±0.05° C. (0.1° F.)
7. Transition span (color to clear)—about 0.3° C. (0.6° F.)
8. Hysteresis or delay (aka time that temperature is above or below trigger temperature before thermochromic liquid crystalline material transitions)—30 seconds

The thermochromic liquid crystalline material is designed to reflect a green wavelength of light at temperatures below about 100° F. (i.e., appear green) and become transparent at temperatures above about 100° F. The thermochromic liquid crystalline material may be altered to reflect other parts of the visible light spectrum such as red or blue and to function at alternative temperature set points ranging from about 99.5° F. to about 102° F. Other parameters such as the delay, accuracy or precision can deviate from what is specified above and the sensor may also satisfactorily function as a skin temperature measurement device. Alternatively chiral thermochromic liquid crystal formula such as those that are commercially available from Liquid Crystal Resource, LLC can also be used.

The thermochromic liquid crystalline material may be applied to the substrate at uniform thickness in, for example, a circular pattern (other patterns, such as squares, triangles, etc. will work as well) 1.5 cm in diameter. The thermochromic liquid crystalline material is disposed on the underlayment layer in such a way that when the overlayment layer is placed over the thermochromic liquid crystalline material, the material covers a circular area having about a 1.5 cm diameter on the underlayment layer as shown in FIG. 9. The indicia 63, particularly the smiling face graphical indicia of this instance, is printed with black ink on the overlayment layer and an additional frowning face in green ink where the green ink matches the green of the thermochromic liquid crystalline material. The initial appearance of the graphical indicia 63 is a black printed smiling face on the overlayment layer, which is placed over the green thermochromic liquid crystalline material. Those areas of the graphical indicia that are unprinted with black ink appear transparent initially over the thermochromic liquid crystalline material which is green in color. As designed, this sensor will change in appearance from the smiling face graphical indicia 63 of FIG. 9 to the frowning face graphical indicia 63 of the same FIG. 9 when a skin temperature of greater than about 100° F. is detected. This change in the graphical indicia is effected by the green color of the thermochromic liquid crystalline material changing to a transparent and uncolored appearance and revealing a printed black surface of the underlayment layer.

The overlayment layer is adhesively, ultrasonically, and/or heat sealed to the underlayment layer in such a way that the thermochromic liquid crystalline material is undisturbed. The seal must be complete to prevent the degradation of the thermochromic liquid crystalline material due to long term exposure to oxygen. In addition, the seal protects the temperature sensing material from being contaminated with impurities that can negatively impact their performance. The substrate may be sealed to the cover using adhesives, heat sealing, clamping, ultrasonic bonding, or any other suitable method. The sensor is then fastened above or below the topsheet of the diaper using any common bonding technique such as adhesives, ultrasonics, heat seals, mechanical fasteners, and such. It is fastened to the topsheet on the interior surface of the article as shown in FIGS. 1-3, with the center of the sensor located between a reasonable amount (e.g., 4-5 cm for Pampers size 2) below the front edge of the disposable diaper in the front waist region.

### Example 2

A diaper of the present invention is made as described in Example 1 but the following changes are made. The circular sensor of Example 1 is replaced with a bar shaped or bar type sensor that is detailed as follows. The width of the rectangular sensor is about 5 cm and a length is about 1.8 cm. (It should be understood that the width of the sensor runs along the transverse width of the article and likewise with the length of the sensor.) The sensor of this article indicates when the skin temperature of the wearer is within any of three temperature ranges by having three distinct regions within it as depicted in FIGS. 5, 6, 11A, 11B, and 11C. The thermochromic liquid crystalline material comprises a plurality of chiral thermochromic liquid crystalline formulations (e.g., three) where each formulation is temperature responsive in different temperature ranges from the other formulations. Each area where a thermochromic liquid crystalline material is disposed may be referred to as a measurement region 65. Such regions may be referred to consecutively depending on the number of regions present in a sensor. Suitable substitutions of cholesteric thermochromic liquid crystalline materials may be made as well. The thermochromic liquid crystalline material, which is operative in the temperature range of from about 95° F. to about 100° F. and is disposed to the left of a first separator in a bar type sensor, i.e., a first measurement region 65, is commercially available from LCR as G95F5W with a change from red to blue at about 100° F. The thermochromic liquid crystalline material which is operative in the temperature range of from about 99° F. to about 101° F. and is disposed in a second measurement region 65 in between the first and a second separator in the bar type sensor, is commercially available from LCR as G99F2W with a change from red to blue occurring at about 101° F. The thermochromic liquid crystalline material of a third measurement region 65, which is operative in the temperature range of from about 101° F. to about 105° F. and is disposed in between the second separator 59 and a right end or edge of the bar type sensor, is commercially available from LCR as G101F4W with a change from red to blue occurring at about 105° F. Each of the thermochromic liquid crystalline material formulations reflect in progression from red to blue all colors of the light spectrum over the specific temperature range and are clear at all other temperatures. These thermochromic liquid crystalline material formulations are each uniformly slot coated onto the underlayment layer in the dimensions mentioned earlier to form these measurement regions. Beforehand, however, the underlayment layer should be printed with an inert black non-solvent based paint so that it is opaque in appearance. Other thermochromic liquid crystalline material formulations may be used that cover temperature ranges of from about 90° F. to about 110° F.

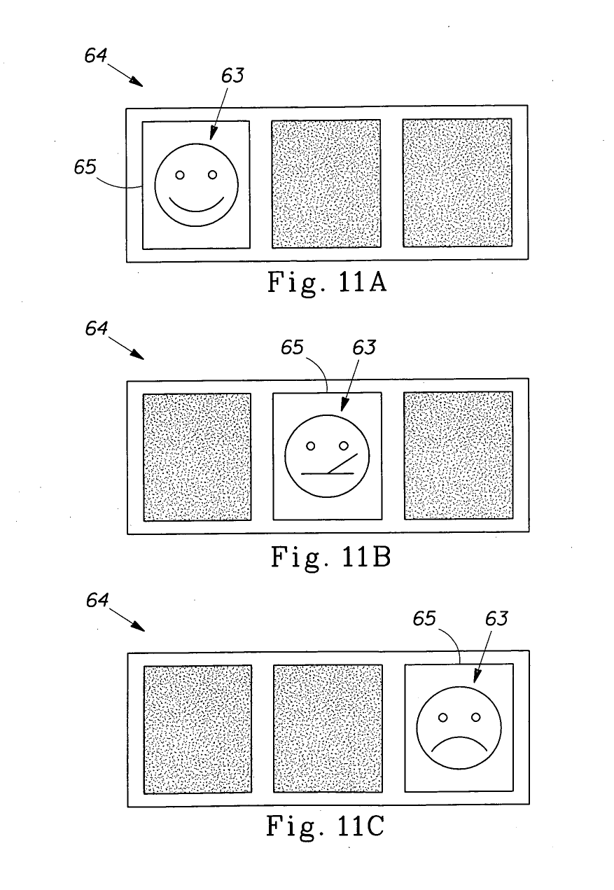

In FIGS. 11A, 11B, and 11C, the bar type sensor is shown where the graphical indicia change during wear according to the body temperature of the wearer. In FIG. 11A, in the first measurement region 65 of the sensor, a smiling face graphical indicia 63 is printed in black ink on an underside of the overlayment layer. In FIG. 11B, in the second measurement region 65, a neutral face serves as the graphical indicia and this image is printed in black ink on the underside of the overlayment layer. In FIG. 11C, in the third measurement region 65, a frowning face graphical indicia is printed in black ink on the underside of the overlayment layer. The overlayment layer is about 5 cm wide, about 1.8 cm long, about 25 μm to about 50 μm thick and also comprises a bi-axially oriented polypropylene. This layer is placed over the top of the thermochromic liquid crystalline material and the underlayment layer and is sealed as detailed in Example 1. The bar type sensor is then adhesively bonded above or below the interior surface of the back waist region of an absorbent article of Example 1.

### Example 3

A diaper with a sensor according to the present invention is made such that the sensor is capable of indicating a temperature trend. The sensor is adhesively or mechanically fastened above or below the topsheet in the front waist region such that the sensor contacts the wearer's abdominal skin surface. The sensor is constructed as detailed in Example 1.

Here, the sensor is rectangular in shape, having a width of about 3.5 cm. and a length of about 1.8 cm. (It should be understood that the width of the sensor runs along the transverse width of the article and likewise with the length of the sensor.) Other shapes may be employed, e.g., rectangular with rounded corners, circular, oval, etc. While the ideal design would have an area of about 6.25 cm² other designs using an area between 2 cm² and 25 cm² may also be effective.

In this instance, the sensor signals an output reading with a thermochromic liquid crystal layer which serves to show a “moving line” along the sensor as the indicated temperature increases. This thermochromic liquid crystalline material is known as L-314, and is commercially available from LCR, LLC of Chicago, Ill. L-314 contains a temperature sensitive liquid crystal formula that, across its span, has a continuously changing temperature set point where the liquid crystal reflects the visible spectrum of light. This creates the effect of a rainbow colored line segment moving up and/or down or across the span of applied thermochromic liquid crystalline material as the detected temperature increases and decreases. The sensor with this thermochromic liquid crystalline material is made as described in Example 1. The liquid crystal should be applied to the underlayment layer at a uniform thickness in a rectangular or slot coat pattern 15 mm in length and 7 mm width. The resulting sensor appears in FIG. 12A, 12B, and 12C. FIG. 12A shows where the indicated temperature is normal. FIG. 12B shows where the sensor indicates that the temperature is somewhat abnormally high and should be checked. FIG. 12C depicts the same sensor where the temperature that is being detected by the wearer is at the point of fever.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments and/or individual features of the present invention have been illustrated and described, it would be obvious to those skilled in the art that various other changes and modifications can be made without departing from the spirit and scope of the invention. Further, it should be apparent that all combinations of such embodiments and features are possible and can result in preferred executions of the invention. Therefore, the appended claims are intended to cover all such changes and modifications that are within the scope of this invention.

What is claimed is:

1. A disposable absorbent article suitable for receiving and containing bodily exudates of a wearer, the article comprising a front waist region, a back waist region and a crotch region disposed between the front and back waist regions, each region having both an interior surface and an exterior surface and wherein the article further comprises a sensor comprising one or more materials selected from the group consisting of thermochromic inks, thermochromic dyes, thermochromic liquid crystalline materials, and combinations thereof such that the sensor is fastened above or below an interior surface of any one of the regions and contacts a wearer's skin when worn.

2. The article of claim 1, wherein the sensor is fastened to one or more of the interior surfaces by a method selected from the group consisting of adhesion, hook and loop fastening, sewing, clamping, magnetism, binding by an outer layer, heat sealing, ultrasonic bonding, and combinations thereof.

3. The article of claim 1, wherein the thermochromic material changes color to indicate a change in human body temperature wherein the change indicates a condition selected from the group consisting of a fever, a hypothermic state, or a combination thereof.

4. The article of claim 1, wherein the sensor comprises an underlayment layer that is disposed beneath the thermochromic liquid crystalline material.

5. The article of claim 4, wherein the underlayment layer comprises a photochromic ink.

6. The article of claim 1, wherein the sensor is covered with an overlayment layer.

7. The article of claim 6, wherein the overlayment layer is at least partially translucent or transparent.

8. The article of claim 6, wherein the overlayment is printed to form one or more indicia.

9. The article of claim 1, wherein the sensor is stiffened to prevent shearing by applying a stiff underlayment layer or by applying stiffening agents to the underside of the underlayment layer or the diaper area.

10. The article of claim 1, wherein the sensor is fastened above or below the front waist region, wherein the sensor is in contact with the wearer's abdomen.

11. The article of claim 1, wherein the sensor comprises wells formed by patterned sealing bonds.

12. The article of claim 1, wherein the sensor comprises one or more indicia useful for indicating an elevated human body temperature the indicia selected from the group consisting of linguistic indicia, graphical indicia, iconic indicia, numerical indicia, color indicia, and combinations thereof.

13. The article of claim 12, wherein the one or more indicia comprises an overlayment that is at least partially transparent such that the thermochromic liquid crystalline material is visible through the transparent overlayment portion.

14. The article of claim 1, wherein the thermochromic liquid crystalline material is encapsulated in gelatin-based microcapsules.

15. The article of claim 1, wherein the sensor contains at least one additional thermochromic liquid crystalline material that is physically separated from the first thermochromic liquid crystalline material within the sensor.

16. The article of claim 1, wherein the material is applied in application method selected from the group consisting of spraying, printing, coating, ultraviolet printing, painting, and combinations thereof.

17. The article of claim 1, wherein the article further comprises one or more additional sensors within the same the region or within another region from the first sensor.

18. The article of claim 1, wherein the sensor is removably fastened.

19. The article of claim 1, wherein the sensor exhibits a delay of at least about 5 seconds.

20. The article of claim 1, wherein the sensor is fastened onto a flap located on the interior surface and wherein the sensor is capable of measuring skin temperature and environmental temperature.

21. The article of claim 1, wherein the sensor is covered by a thin film or nonwoven.

22. A method of visually detecting a wearer's health status, the method comprising the steps of:
a. providing a caregiver with a disposable absorbent article suitable for receiving and containing bodily exudates of a wearer, the article comprising a front waist region, a back waist region and a crotch region disposed between the front and back waist region, each region an interior surface and an exterior surface and wherein the article further comprises a sensor comprising one or more materials selected from the group consisting of thermochromic inks, thermochromic dyes, thermochromic liquid crystalline materials, and combinations thereof such that the sensor is fastened to an interior surface of any one of the regions;
b. donning the article onto a wearer; and
c. instructing the caregiver to visually monitor the wearer's health status via the appearance of the sensor.

23. A kit for visually detecting a wearer's health status, the kit comprising:
a. one or more disposable absorbent articles suitable for receiving and containing bodily exudates of a wearer, each of the articles comprising a front waist region, a back waist region and a crotch region disposed between the front and back waist regions, each region an interior surface and an exterior surface and wherein each of the articles further comprise a sensor comprising one or more materials selected from the group consisting of thermochromic inks, thermochromic dyes, thermochromic liquid crystalline materials, and combinations thereof such that the sensor is fastened to an interior surface of any one of the regions; and
b. a set of instructions for a caregiver or the wearer to visually detect the status of an infant wearer's health.

24. The kit of claim 23, wherein the kit further comprises a system for recording and tracking temperature of the infant wearer
